(19) United States
(12) Patent Application Publication
(10) Pub. No.: US 2024/0277482 A1
(43) Pub. Date: Aug. 22, 2024

(54) MENISCUS IMPLANTS AND METHODS OF MAKING SAME

(71) Applicant: Shu-Tung and Alice Li Foundation Inc., Oakland, NJ (US)

(72) Inventors: Shu-Tung Li, Franklin Lakes, NJ (US); Wei Chang, Oakland, NJ (US)

(73) Assignee: Shu-Tung and Alice Li Foundation Inc., Oakland, NJ (US)

(21) Appl. No.: 18/582,198

(22) Filed: Feb. 20, 2024

Related U.S. Application Data
(60) Provisional application No. 63/486,101, filed on Feb. 21, 2023.

(51) Int. Cl. A61F 2/38; A61F 2/30; A61L 27/20; A61L 27/24; A61L 27/56

(52) U.S. Cl. CPC A61F 2/3872; A61F 2/3094; A61L 27/20; A61L 27/24; A61L 27/56

(57) ABSTRACT

The present invention relates to a glycosaminoglycan impregnated biopolymer-based scaffold implant for repair and regeneration of damaged and diseased human menisci. The biopolymer can be any suitable natural or genetically engineered biopolymers. This scaffold implant has several advantages over prior art implants including higher biomechanical strength and lower surface friction. A method of making the scaffold implant is also disclosed.

### CROSS-REFERENCE TO RELATED APPLICATIONS

This application claims priority under 35 U.S.C. § 119(e) to U.S. Provisional Patent Application No. 63/486,101, filed Feb. 21, 2023. The foregoing application is incorporated by reference herein in its entirety.

### FIELD OF THE INVENTION

This invention relates generally to improved meniscus implants for repairing injured or diseased meniscus and methods of making same.

### BACKGROUND OF THE INVENTION

Menisci are two crescent-shaped and slightly concaved wedges of fibrocartilage, situated between the femoral condyles and tibia plateau, providing crucial biomechanical functions to the knee joint. Damage to menisci will result in abnormal joint mechanics, which is a prelude to the development of osteoarthritis.

One of the major biopolymeric components in the extracellular matrix of menisci is type I collagen, which serves the structural supporting function of menisci in the knee joint of the body. As type I collagen is homologous among all mammals, many implants made of type I collagen from animal sources are on the market today for tissue repair and regeneration. Type I collagen, in combination with a small amount of glycosaminoglycan (GAG), was used to make collagen implants for meniscus repair. The implant was made with randomly oriented type I collagen GAG composite fibers. The product has been in clinical use both in Europe and in the United States for more than 15 years, only with limited success, primarily due to insufficient mechanical strength to support in vivo functions as a temporary meniscus substitute during the period of healing. As a result, a long rehabilitation program is required to prevent re-tear of the implant prematurely. Even then, tear or re-tear still occurs, and a second surgery is required to correct or replace the implant.

Therefore, there is a strong need for an improved meniscus implant for repairing injured or diseased meniscus.

### SUMMARY OF THE INVENTION

This disclosure addresses the above need in a number of aspects. In one aspect, this disclosure provides a scaffold implant for repairing injured or diseased meniscus. In some embodiments, the scaffold implant comprises biopolymer fibers formed of a biopolymer impregnated with glycosaminoglycan, wherein greater than 30% by weight of the biopolymer fibers are oriented along a circumferential direction, and wherein the scaffold implant has characteristics comprising: an average density from 0.14 g/cm³ to 0.3 g/cm³, a compartmental density of an inner rim ranging from 0.15 g/cm³ to 0.35 g/cm³, a compartmental density of an outer rim ranging from 0.1 g/cm³ to 0.25 g/cm³, a pore volume from 60% to 90%, and a surface friction coefficient from 0.05 to 2.0.

In some embodiments, the scaffold implant has one or more or all of the following characteristics: an inner rim tensile strength of 500 N/cm² to 1000 N/cm², an outer rim tensile strength of 100 N/cm² to 500 N/cm², an inner rim pore size of 40 μm to 150 μm, an outer rim pore size of 50 μm to 250 μm, an inner rim pore volume of 70% to 85%, an outer rim pore volume of 80% to 95%, and a suture retention of 20 N to 40 N.

In some embodiments, the biopolymer comprises collagen. In some embodiments, the collagen comprises a type I collagen, a type II collagen, a type III collagen, or a combination thereof. In some embodiments, the collagen comprises a type I collagen or a type II collagen. In some embodiments, the collagen is derived from a human or a non-human animal, or generated from a genetically modified organism.

In some embodiments, the glycosaminoglycan is hyaluronic acid (HA), chondroitin sulfate, chitosan, alginic acid, or a combination thereof. In some embodiments, the scaffold implant comprises about 0.1% to about 5% by weight of hyaluronic acid. In some embodiments, the scaffold implant comprises about 0.5% to about 3% by weight of hyaluronic acid. In some embodiments, the hyaluronic acid has a molecular weight ranging from about 0.1×10⁶ Daltons to about 3.0×10⁶ Daltons. In some embodiments, the hyaluronic acid is impregnated into the scaffold implant via injection or soaking in a solution of the hyaluronic acid after a scaffold of the scaffold implant has been engineered.

In some embodiments, greater than 50%, 60%, or 70% of the biopolymer fibers are oriented along a circumferential direction. In some embodiments, the friction coefficient is from about 0.1 to about 1.0.

In some embodiments, the scaffold implant is crosslinked by an aldehyde-based crosslinking agent. In some embodiments, the aldehyde-based crosslinking agent comprises formaldehyde. In some embodiments, residuals of the aldehyde-based crosslinking agent are removed by water or a Tris solution.

In some embodiments, the scaffold implant further comprises one or more bioactive elements. In some embodiments, the bioactive elements are autologous or allogenous. In some embodiments, the bioactive elements comprise platelet rich plasma (PRP), cells, bioactive molecules, or a combination thereof. In some embodiments, the bioactive elements are allogenous and comprise bioactive molecules or stem cells. In some embodiments, the bioactive elements comprise a human recombinant bioactive molecule or a drug.

In another aspect, this disclosure also provides a method for making a scaffold implant. In some embodiments, the method comprises: (a) preparing a dispersion comprising a biopolymer; (b) reconstituting the biopolymer in the dispersion into biopolymer fibers; (c) aligning the biopolymer fibers onto a rotating mandrel to form aligned biopolymer fibers; (d) dehydrating the biopolymer fibers partially in a dehydration chamber; (e) placing the aligned biopolymer fibers in a first mold of a defined dimension; (f) applying a first weight to the biopolymer fibers to dehydrate the biopolymer fibers and form a first partially dehydrated biopolymer scaffold matrix; (g) removing the first weight and applying a second weight to the first partially dehydrated biopolymer scaffold matrix using a second mold to control a regional density of the biopolymer fibers and form a second partially dehydrated biopolymer scaffold matrix; (h) freeze-drying the second partially dehydrated biopolymer scaffold matrix to obtain a freeze-dried scaffold matrix; (i) crosslinking the freeze-dried scaffold matrix; and (k) sizing the freeze-dried scaffold matrix to obtain a scaffold implant.

In some embodiments, the method further comprises incorporating hyaluronic molecules into the scaffold implant after the step of sizing.

In some embodiments, the biopolymer comprises collagen. In some embodiments, the collagen comprises a type I collagen, a type II collagen, a type III collagen, or a combination thereof.

Also within the scope of this disclosure is a meniscus scaffold implant prepared according to the method described herein.

In yet another aspect, this disclosure further provides a method of treating a joint of a subject using a scaffold implant. In some embodiments, the method comprises providing a scaffold implant described herein, and placing the scaffold implant to the joint of the subject.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, 1D, 1E, and IF show an example process of making a collagen-based scaffold meniscus implant. FIG. 1A shows that coacervated collagen fibers were first elongated in a tube before they were slowly winded onto the rotating mandrel 11. FIG. 1B shows two bars 16 were positioned at the side of the mandrel. FIG. 1C shows that the bars and the weight were used to compress the collagen fibers for enhancing fiber orientation and partial dehydration. FIG. 1D shows that the first weight was applied from the weight element to slowly dehydrate the collagen fibers. FIG. 1E shows that a dehydrated, molded, fiber-oriented meniscus scaffold was subjected to a second molding process. FIG. 1F shows that formaldehyde residuals were removed by a Tris solution and water. The final crosslinked collagen was cut in half to provide two separate collagen menisci.

FIGS. 2A and 2B show scanning electron microscope (SEM) images and Fast Fourier Transform (FFT) analysis: randomly oriented fibers (FIG. 2A) and aligned oriented fibers (FIG. 2B).

FIGS. 3A, 3B, and 3C show an example of an in-house pendulum that simulates the knee joint mechanics: front view (FIG. 3A), side view (FIG. 3B), and coefficient of friction (FIG. 3C).

FIGS. 4A, 4B, and 4C show durability testing: an in-house device for durability test (FIG. 4A), in phosphate-buffered saline (PBS) after 20 hours with 72,000 resolutions (FIG. 4B), and in hyaluronic acid (HA) after 20 hours with 72,000 resolutions (FIG. 4C).

### DETAIL DESCRIPTION OF THE INVENTION

The existing collagen-based scaffold meniscus implants, such as the implant product initially marketed in Europe by ReGen Biologics and later marketed in the United States by Stryker, have mixed clinical outcomes. Even though the implant supports new tissue growth, there are several drawbacks to using it, as summarized below.

First, the collagen fibers in the implant are randomly oriented in the product resulting in a low tensile strength along the circumferential stress direction. Second, the density of the implant is not optimized to balance pore size with respect to overall strength. Since the pore volume (the empty space of the implant) is at about 90% (average density about 0.14 g/cm³), the overall mechanical properties are not optimal for functioning as a meniscus scaffold. Further, since the implant serves as a temporary meniscus substitute, the rate of resorption and the rate of new tissue deposition and subsequent tissue remodeling are not adequately balanced, leading to premature re-tear and failure. Finally, the friction coefficient is significantly higher than the surface of the native meniscus, which can cause shear-induced damage to the implant.

Due to the above drawbacks, a long rehabilitation program (e.g., 3-6 months) is required for the product. As a result, many cases of re-tear surgeries were performed, resulting partly from patients not in full compliance with rehab protocols. In addition, there were many cases of incomplete regeneration due to loss of implant parts, resulting in smaller sizes of regenerated menisci, i.e., indicating mechanical insufficiency.

This disclosure addresses the above drawbacks in various aspects by providing a novel scaffold implant for repairing or replacing injured or diseased human meniscus. First, in various embodiments of the disclosed scaffold implant, collagen fibers are largely oriented along the direction of stress, leading to a multiple-fold increase of tensile strength, minimizing the potential tear of the scaffold implant during important healing periods.

Second, the density of the scaffold implant is redistributed to increase the suture retention strength without compromising other design requirements (e.g., pore structure), and to provide higher overall mechanical strength for in vivo stability.

Third, the surface in contact with the femoral condyle will be continuously lubricated with an agent such as hyaluronic acid, a key component of the synovial fluid in the knee joint. Varied sizes of hyaluronic acid molecules are impregnated into the scaffold implant for controlled release of hyaluronic acid molecules to the surface at different rates, providing a prolonged period of surface protection from potential shear stress-induced surface damage.

Fourth, various bioactive elements can be incorporated in situ at the point of surgery to facilitate and enhance the rate of healing such that the rehab program can be more aggressively planned.

The scaffold implants according to this disclosure are described in greater detail below.

### Scaffold Implants

In one aspect, this disclosure provides a scaffold implant for repairing injured or diseased meniscus. A scaffold implant is a cell-free structure that can be used to create new functional tissues for medical purposes. Scaffolds are often made of polymeric biomaterials, such as 3D biomimetic materials that provide structural support and an artificial extracellular matrix for cells.

Meniscus is a crescent-shaped pad of fibrocartilage found within certain joints, primarily the knee. There are two menisci in each knee, located between the femur (thighbone) and the tibia (shinbone). They function as shock absorbers and stabilizers, distributing weight evenly across the joint and preventing excessive bone-on-bone contact.

In some embodiments, greater than 30% (i.e., 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, or more) by weight of the biopolymer fibers are oriented along a circumferential direction.

As used herein, the term “circumferential direction” refers to a direction that follows around the curve or circumference of a scaffold implant. Taking a string and wrapping it around the scaffold implant to trace its outline, the path traced by the string represents the circumferential direction of the scaffold implant.

In some embodiments, greater than 50% (i.e., 50%, 52%, 54%, 56%, 58%, 60%, 62%, 64%, 66%, 68%, 70%, 72%, 74%, 76%, 78%, 80%, 82%, 84%, 86%, 88%, 90%, or more) by weight of the biopolymer fibers are oriented along a circumferential direction. In some embodiments, greater than 60% (i.e., 60%, 62%, 64%, 66%, 68%, 70%, 72%, 74%, 76%, 78%, 80%, 82%, 84%, 86%, 88%, 90%, or more) by weight of the biopolymer fibers are oriented along a circumferential direction. In some embodiments, greater than 70% (i.e., 70%, 72%, 74%, 76%, 78%, 80%, 82%, 84%, 86%, 88%, 90%, or more) by weight of the biopolymer fibers are oriented along a circumferential direction.

In some embodiments, the scaffold implant has an average density from 0.14 g/cm³ to 0.3 g/cm³ (e.g., 0.14 g/cm³, 0.15 g/cm³, 0.16 g/cm³, 0.17 g/cm³, 0.18 g/cm³, 0.19 g/cm³, 0.2 g/cm³, 0.21 g/cm³, 0.22 g/cm³, 0.23 g/cm³, 0.24 g/cm³, 0.25 g/cm³, 0.26 g/cm³, 0.27 g/cm³, 0.28 g/cm³, 0.29 g/cm³, or 0.3 g/cm³).

In some embodiments, the scaffold implant has a compartmental density of an inner rim ranging from 0.15 g/cm³ to 0.35 g/cm³ (e.g., 0.15 g/cm³, 0.16 g/cm³, 0.17 g/cm³, 0.18 g/cm³, 0.19 g/cm³, 0.2 g/cm³, 0.21 g/cm³, 0.22 g/cm³, 0.23 g/cm³, 0.24 g/cm³, 0.25 g/cm³, 0.26 g/cm³, 0.27 g/cm³, 0.28 g/cm³, 0.29 g/cm³, 0.3 g/cm³, 0.31 g/cm³, 0.32 g/cm³, 0.33 g/cm³, 0.34 g/cm³, or 0.35 g/cm³).

In some embodiments, the scaffold implant has a compartmental density of an outer rim ranging from 0.1 g/cm³ to 0.25 g/cm³ (e.g. 0.1 g/cm³, 0.11 g/cm³, 0.12 g/cm³, 0.13 g/cm³, 0.14 g/cm³, 0.15 g/cm³, 0.16 g/cm³, 0.17 g/cm³, 0.18 g/cm³, 0.19 g/cm³, 0.2 g/cm³, 0.21 g/cm³, 0.22 g/cm³, 0.23 g/cm³, 0.24 g/cm³, or 0.25 g/cm³).

As used herein, the term “inner rim” refers to the inner edge of the scaffold implant. It is the smaller of the two inner and outer edges, located closer to the center of the scaffold implant. As used herein, the term “outer rim” refers to the outer edge of the scaffold implant that is farther away from the center of the scaffold implant, encircling the inner rim. It is typically larger than the inner rim.

In some embodiments, the scaffold implant has a pore volume from 60% to 90% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%). As used herein, the term “pore volume” refers to the total volume of all the pores (e.g., openings, air pockets) within a scaffold implant, expressed as a percentage of the total volume of the scaffold implant. It is essentially the amount of empty space inside the scaffold implant.

In some embodiments, the scaffold implant has a surface friction coefficient from 0.05 to 2.0 (e.g., 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, or 2). In some embodiments, the friction coefficient is from about 0.1 to about 1.0 (e.g., 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1).

As used herein, the term “surface friction coefficient” or “coefficient of friction (μ)” refers to a dimensionless quantity that indicates the magnitude of frictional resistance between two surfaces in contact. In simpler terms, it tells you how “sticky” or slippery the surfaces are with respect to each other. Materials with higher coefficients of friction experience greater frictional forces, meaning more force is needed to overcome friction and make them slide past each other. There are two main types of coefficients of friction: static coefficient of friction (μs) and kinetic coefficient of friction (μk). The static coefficient of friction (μs) measures the friction force needed to start movement between two surfaces that are initially at rest. The kinetic coefficient of friction (μk) measures the friction force acting between two surfaces that are already in motion. It is usually slightly lower than the static coefficient.

In some embodiments, the scaffold implant has one or more or all of the following characteristics: an inner rim tensile strength of 500 N/cm² to 1000 N/cm² (e.g., 500, 520, 540, 560, 580, 600, 620, 640, 660, 680, 700, 720, 740, 760, 780, 800, 820, 840, 860, 880, 900, 920, 940, 960, 980, or 1000 N/cm²), an outer rim tensile strength of 100 N/cm² to 500 N/cm² (e.g., 100, 120, 140, 160, 180, 200, 220, 240, 260, 280, 300, 320, 340, 360, 380, 400, 420, 440, 460, 480, or 500 N/cm²), an inner rim pore size of 40 μm to 150 μm (e.g., 40, 45, 50, 55, 60, 65, 70, 75, 80, 85, 90, 95, 100, 105, 110, 115, 120, 125, 130, 135, 140, 145, or 150 μm), an outer rim pore size of 50 μm to 250 μm (e.g., 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 μm), an inner rim pore volume of 70% to 85% (e.g., 70%, 71%, 72%, 73%, 74%, 75%, 76%, 77%, 78%, 79%, 80%, 81%, 82%, 83%, 84%, 85%), an outer rim pore volume of 80% to 95% (e.g., 80%, 81%, 82%, 83%, 84%, 85%, 86%, 87%, 88%, 89%, 90%, 91%, 92%, 93%, 94%, or 95%,), and/or a suture retention of 20 N to 40 N (e.g., 20 N, 21 N, 22 N, 23 N, 24 N, 25 N, 26 N, 27 N, 28 N, 29 N, 30 N, 31 N, 32 N, 33 N, 34 N, 35 N, 36 N, 37 N, 38 N, 39 N, or 40 N).

In some embodiments, the scaffold implant has an inner rim tensile strength of 500 N/cm² to 1000 N/cm² (e.g., 500, 520, 540, 560, 580, 600, 620, 640, 660, 680, 700, 720, 740, 760, 780, 800, 820, 840, 860, 880, 900, 920, 940, 960, 980, or 1000 N/cm²).

In some embodiments, the scaffold implant has an outer rim tensile strength of 100 N/cm² to 500 N/cm² (e.g., 100, 120, 140, 160, 180, 200, 220, 240, 260, 280, 300, 320, 340, 360, 380, 400, 420, 440, 460, 480, or 500 N/cm²).

In some embodiments, the scaffold implant has an inner rim pore size of 40 μm to 150 μm (e.g., 40, 45, 50, 55, 60, 65, 70, 75, 80, 85, 90, 95, 100, 105, 110, 115, 120, 125, 130, 135, 140, 145, or 150 μm).

In some embodiments, the scaffold implant has an outer rim pore size of 50 μm to 250 μm (e.g., 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 μm).

In some embodiments, the scaffold implant has an inner rim pore volume of 70% to 85% (e.g., 70%, 71%, 72%, 73%, 74%, 75%, 76%, 77%, 78%, 79%, 80%, 81%, 82%, 83%, 84%, 85%).

In some embodiments, the scaffold implant has an outer rim pore volume of 80% to 95% (e.g., 80%, 81%, 82%, 83%, 84%, 85%, 86%, 87%, 88%, 89%, 90%, 91%, 92%, 93%, 94%, or 95%,).

In some embodiments, the scaffold implant has and a suture retention of 20 N to 40 N (e.g., 20 N, 21 N, 22 N, 23 N, 24 N, 25 N, 26 N, 27 N, 28 N, 29 N, 30 N, 31 N, 32 N, 33 N, 34 N, 35 N, 36 N, 37 N, 38 N, 39 N, or 40 N).

In some embodiments, the scaffold implant may include biopolymer fibers formed of a biopolymer impregnated with glycosaminoglycan. As used herein, the term “glycosaminoglycan” or “GAG” refers to long, unbranched polysaccharides consisting of repeating disaccharide units. The repeating unit consists of a hexose (six-carbon sugar) or a hexuronic acid, linked to a hexosamine (six-carbon sugar containing nitrogen) and pharmaceutically acceptable salts thereof. Members of the GAG family vary in the type of hexosamine, hexose, or hexuronic acid unit they contain, such as, e.g., glucuronic acid, iduronic acid, galactose, galactosamine, glucosamine) and may also vary in the geometry of the glycosidic linkage. Non-limiting examples of glycosaminoglycans include chondroitin sulfate, dermatan sulfate, keratan sulfate, and hyaluronan. Non-limiting examples of acceptable salts of glycosaminoglycans include sodium salts, potassium salts, magnesium salts, calcium salts, and combinations thereof.

In some embodiments, the glycosaminoglycan is hyaluronic acid, chondroitin sulfate, chitosan, alginic acid, or a combination thereof.

In some embodiments, the scaffold implant may include about 0.1% to about 5% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5%) by weight of hyaluronic acid, a hyaluronic acid polymer, or a derivative thereof.

In some embodiments, the scaffold implant may include about 0.5% to about 3% (e.g., 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, or 3%) by weight of hyaluronic acid, a hyaluronic acid polymer, or a derivative thereof.

Hyaluronan, also known as hyaluronic acid (HA) is a non-sulfated glycosaminoglycan that is distributed widely throughout the human body in connective, epithelial, and neural tissues. Hyaluronan is abundant in the different layers of the skin, where it has multiple functions such as, e.g., to ensure good hydration, to assist in the organization of the extracellular matrix, to act as a filler material; and to participate in tissue repair mechanisms.

As used herein, the term “hyaluronic acid polymer” is synonymous with “HA polymer”, “hyaluronic acid polymer,” and “hyaluronate polymer” refers to an anionic, non-sulfated glycosaminoglycan polymer comprising disaccharide units, which themselves include D-glucuronic acid and D-N-acetylglucosamine monomers, linked together via alternating β-1,4 and β-1,3 glycosidic bonds and pharmaceutically acceptable salts thereof. Hyaluronan polymers can be purified from animal and non-animal sources. Polymers of hyaluronan can range in size from about 5,000 Da to about 20,000,000 Da. Non-limiting examples of pharmaceutically acceptable salts of hyaluronan include sodium hyaluronan, potassium hyaluronan, magnesium hyaluronan, calcium hyaluronan, and combinations thereof.

As used herein, the term “derivative” refers to a structurally similar compound that retains sufficient functional attributes of the identified analog. The derivative may be structurally similar because it is lacking one or more atoms, substituted, a salt, in different hydration/oxidation states, or because one or more atoms within the molecule are switched, such as, but not limited to, replacing an oxygen atom with a sulfur atom or replacing an amino group with a hydroxy group. Derivatives may be prepared by any variety of synthetic methods or appropriate adaptations presented in synthetic or organic chemistry textbooks, such as those provided in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze, hereby incorporated by reference.

In some embodiments, the hyaluronic acid has a molecular weight ranging from about 0.1×10⁶ Daltons to about 3.0×10⁶ Daltons (e.g., 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, or 3×10⁶ Daltons).

In some embodiments, hyaluronic acid is impregnated into the scaffold implant via injection or soaking in a solution of the hyaluronic acid after a scaffold of the scaffold implant has been engineered.

As used herein, the term “impregnation” refers to permeation or saturation of something with another substance. In some embodiments, the biopolymer fibers (e.g., collagen fibers) can be impregnated with one or more glycosaminoglycans (e.g., hyaluronic acid) in various ways, such as soaking or injection.

In some embodiments, the biopolymer may include collagen. The term “collagen,” as used herein, refers to a natural protein having the molecular structure known as atelocollagen, telocollagen, tropocollagen, procollagen, polymeric collagen, fibrillar collagen, electrospun collagen, insoluble collagen, soluble collagen, precipitated collagen, or collagen dough. As used herein, the term “native collagen” refers to collagen that retains normal conformation, unlike gelatin, which is collagen that has been irreversibly hydrolyzed. Collagen is the major protein in all mammals. Currently, twenty-eight genetically distinct types of collagens have been discovered. It accounts for about 90% of the total proteins in the body. Fiber-forming collagens (type I, type II, and type III collagen) are homologous among all mammals, and they account for the majority (90%) of the collagen in the body. They are the major proteins in bone (type I collagen), skin (type I and type III collagens), tendon (type I collagen), cartilage (type II collagen), and ligament (type I collagen). Therefore, a large quantity of fiber-forming collagen-rich tissues can be harvested from the abattoirs.

In some embodiments, the collagen may include a type I collagen, a type II collagen, a type III collagen, or a combination thereof. In some embodiments, the collagen may include a type I collagen. In some embodiments, the collagen may include a type II collagen.

In some embodiments, collagen is derived from a human or a non-human animal or generated from a genetically modified organism, such as an animal that is genetically modified to produce collagen.

In some embodiments, the scaffold implant is crosslinked by an aldehyde-based crosslinking agent. An aldehyde-based crosslinking agent can be a chemical compound containing an aldehyde functional group (CHO) used to link different molecules together, forming a more extensive and interconnected network. They primarily target functional groups like amines (NH₂) and hydroxyls (OH) present in various materials, creating covalent bonds between them. Aldehyde-based crosslinking agents can react with functional groups like amines and hydroxyls through a condensation reaction, forming Schiff bases or hemiacetals/acetals, respectively. These bonds are relatively strong and contribute to the crosslinking effect.

In some embodiments, the aldehyde-based crosslinking agent may include formaldehyde, glutaraldehyde, glyoxal, and/or dialdehyde starch. In some embodiments, the aldehyde-based crosslinking agent may include formaldehyde.

In some embodiments, residuals of the aldehyde-based crosslinking agent are removed by water, a solution, or a buffer, such as a Tris solution (e.g., a Tris buffer). A Tris buffer (e.g., TriCl buffer) may have an effective pH range of between 7.1 and 9.1.

In some embodiments, the scaffold implant further may include one or more bioactive elements. As used herein, the term “bioactive elements” refers to naturally occurring chemical or biological components found in living organisms that have the ability to interact with and influence biological processes. They exist in a wide variety and are found in diverse sources, including animals or microorganisms.

In some embodiments, the bioactive elements are autologous or allogenous. In some embodiments, the bioactive elements may include platelet-rich plasma (PRP), cells, bioactive molecules, or a combination thereof. In some embodiments, the bioactive elements are allogenous and may include bioactive molecules or stem cells. In some embodiments, the bioactive elements may include a human recombinant bioactive molecule or a drug.

### Methods of Making Scaffold Implants and Methods of Uses

In another aspect, this disclosure also provides a method for making a scaffold implant. In some embodiments, the method may include: (a) preparing a dispersion comprising a biopolymer; (b) reconstituting the biopolymer in the dispersion into biopolymer fibers; (c) aligning the biopolymer fibers onto a rotating mandrel to form aligned biopolymer fibers; (d) dehydrating the biopolymer fibers partially in a dehydration chamber; (c) placing the aligned biopolymer fibers in a first mold of a defined dimension; (f) applying a first weight to the biopolymer fibers to dehydrate the biopolymer fibers and form a first partially dehydrated (but still wet) biopolymer scaffold matrix; (g) removing the first weight and applying a second weight to the first partially dehydrated biopolymer scaffold matrix using a second mold to control a regional density of the biopolymer fibers and form a second partially dehydrated (but still wet) biopolymer scaffold matrix; (h) freeze-drying the second partially dehydrated biopolymer scaffold matrix to obtain a freeze-dried scaffold matrix; (i) crosslinking the freeze-dried scaffold matrix; and (k) sizing the freeze-dried scaffold matrix to obtain a scaffold implant.

In some embodiments, the press head (the first weight) in the first mold has a steeper curvature which distributes the collagen fibers toward the outer rim. Once the compression is completed, the first mold was replaced by the second mold where the press head (the second weight) has a flatter curvature to redistribute the collagen fibers resulting in an increase of the local density at the outer rim (see Table 1, P 21). The higher the density, the stronger the suture retention strength will be reached.

In some embodiments, the method may further include incorporating hyaluronic molecules into the scaffold implant after the step of sizing.

In some embodiments, the step of sizing may include generating a meniscus from a circular-shaped scaffold implant by cutting the circular-shaped scaffold implant into two halves, each of which is shaped into a meniscus.

The term “dispersion” refers to a dispersed system in which one substance, the dispersed phase (e.g., collagen fiber particles), is distributed in discrete units throughout a second substance (the continuous phase or vehicle). The size of the dispersed phase can vary (e.g., microns in size).

In some embodiments, the biopolymer may include collagen. In some embodiments, the collagen may include a type I collagen, a type II collagen, a type III collagen, or a combination thereof.

Also within the scope of this disclosure is a meniscus scaffold implant prepared according to the method described herein.

In yet another aspect, this disclosure further provides a method of treating a joint of a subject using a scaffold implant. In some embodiments, the method may include providing a scaffold implant described herein, and placing the scaffold implant to the joint of the subject.

The surgical process may include collagen meniscus implant (CMI) surgery if the implant is a synthetic scaffold made of collagen or meniscal scaffold implant surgery (similar to CMI but could encompass other scaffold materials). It may include meniscal repair with a scaffold implant if the implant is used to supplement or reinforce a repair of a damaged meniscus rather than replacing it entirely. The type of implant and procedure will depend on various factors like the severity and location of the meniscus damage, the patient's age and activity level, and the surgeon's preferences. Meniscus implant surgery is typically performed arthroscopically, meaning through small incisions and with the aid of a camera. Recovery time can vary depending on the specific procedure, but it generally involves several months of physical therapy and rehabilitation.

### Additional Definition

To aid in understanding the detailed description of the agents and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, a “subject” refers to a human and a non-human animal. Examples of non-human animals include all vertebrates, e.g., mammals, such as non-human mammals, non-human primates (particularly higher primates), dogs, rodents (e.g., mice or rats), guinea pigs, cats, and rabbits, and non-mammals, such as birds, amphibians, reptiles, etc. In one embodiment, the subject is a human. In another embodiment, the subject is an experimental animal or animal suitable as a disease model.

As used herein, the term “in vitro” refers to events that occur in an artificial environment, e.g., in a test tube or reaction vessel, in cell culture, etc., rather than within a multicellular organism.

As used herein, the term “in vivo” refers to events that occur within a multicellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method may include a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLE

### Example 1

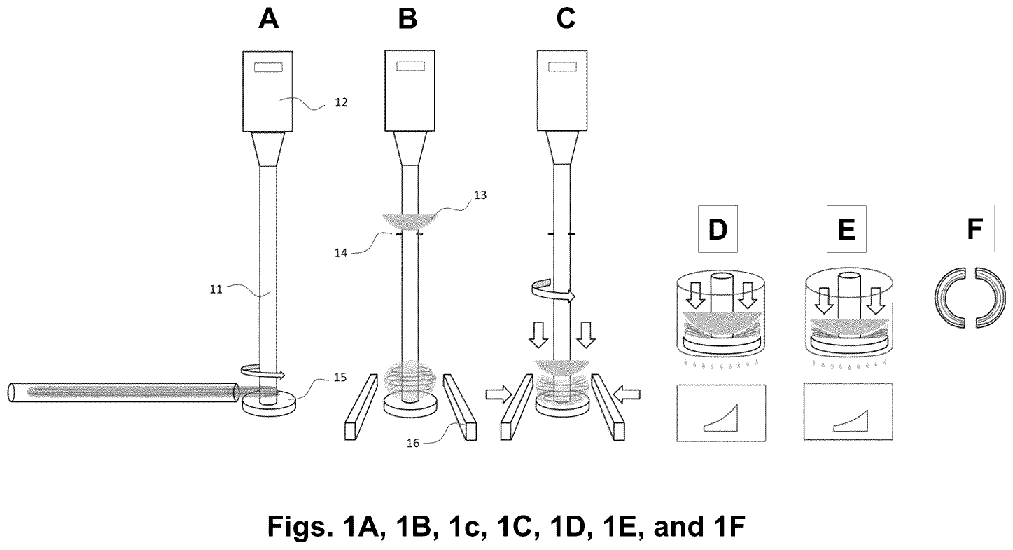

FIG. 1 depicts the processing steps for engineering a scaffold implant using type I collagen fiber material. The coacervated collagen fibers described were first elongated in a tube before they were slowly winded onto rotating mandrel 11 (FIG. 1A). A rotational mandrel 11 was attached to a commercial digital stirrer 12 (Caframo Digital Stirrer, Georgian Bluffs, ON Canada). The speed of rotation can be controlled within the range of interest (e.g., 1-100 RPM). At the bottom of the rotating mandrel is a Delrin base plate 15 for holding the collagen fibers in place. The base plate 15 was made permeable to water to facilitate dehydration. At the top of the mandrel is a cone-shaped stainless-steel weight 13 with a slight concave curvature to create the curvature of the meniscus. The weight element can slide along the mandrel, once released from its fixed position by a pin (element 14). There are two bars 16 positioned at the side of the mandrel (FIG. 1B). The bars and the weight were used to compress the collagen fibers for enhancing fiber orientation and partial dehydration (FIG. 1C).

After the weight reached the base plate, the oriented collagen fibers were then transferred to a 2-step molding system (FIGS. 1D and 1E) for local density improvement. The molding system is made of polycarbonate housing having a dimension that fits with the weight element and the base plate. The first weight was applied from the weight element to slowly dehydrate the collagen fibers (FIG. 1D). The dehydrated, molded, fiber-oriented meniscus scaffold was then subjected to a second molding process shown in FIG. 1E to redistribute the collagen fibers. The final prototype has a higher collagen density at the peripheral side and was removed from the mold for freeze drying in a commercial freeze dryer (Virtis, Gardiner, NY). The freeze-dried scaffold was then crosslinked with formaldehyde. Formaldehyde residuals were removed by a Tris solution and water. The final crosslinked collagen was cut in half to provide two separate collagen menisci (FIG. 1F).

Different molecular weights of hyaluronic acid (HA) molecules (0.1×10⁶ to 3×10⁶ Daltons) were then impregnated into the scaffold via soaking in a hyaluronic acid solution. The total hyaluronic acid content was about 1% of the weight of the scaffold. The hyaluronic acid-incorporated scaffold implant was then dried, packaged, and sterilized.

Although various biopolymeric materials can be used to engineer the extracellular matrix (ECM) scaffold implant, the use of type I collagen was demonstrated in this example. Type I collagen is the main component of the extracellular matrices, including menisci, and is homologous among all mammalian species. It is well-accepted as a material for implant development. Purified type I collagen fibers were first prepared. Methods for collagen isolation and purification have been described previously, e.g., U.S. Pat. Nos. 5,681,353 and 5,735,903, the disclosures of which are incorporated herein by reference. Briefly, tendon from bovine was used as the source for type I collagen. After cleaning and removing the extraneous tissues, the tendon was blocked, frozen, and mechanically disintegrated into small pieces for case of purification. The tendon was extracted in various solvents to remove the non-collagenous moieties, including various acidic and basic moieties (e.g., glycoproteins, GAGs, histones), lipids, DNAs, cell debris, and various chemicals used in the process (e.g., acid, base, salts, alcohols), rendering the purified intact collagen fibers implantable. The purified type I collagen was stored in the freeze-dried form.

An example process of engineering a collagen scaffold implant is described below. A fixed weight of purified collagen fibers prepared above was suspended in a fixed volume of 0.07 M lactic acid for a final collagen content of 0.7% (w/v), pH 2.3 overnight at 4° C. The suspension was subsequently homogenized to reduce the fiber size to fibrils for uniform dispersion using a commercial homogenizer (Silverson, East Longmeadow, MA). pH values of aliquots of the dispersion, containing a fixed amount of collagen to form a final human-size implant with an average density in the range of 0.20-0.30 g/cm³, were then adjusted to the isoelectric point of collagen (about pH 5.5) with 1M NH₄OH to reconstitute the fibers. Upon de-air to remove the trapped air within the reconstituted fibers, the fibers were first oriented and slowly wrapped around a rotating stainless-steel mandrel covered with a polytetrafluoroethylene (PTFE) tubing with an outer diameter (OD) of 2.0 cm (11 in FIG. 1A) at a rotating speed of 5-15 RPM to align the fibers. The partially aligned collagen fibers on the mandrel were then placed at the center of the dehydration element (15 in FIG. 1B). The pin (14 in FIG. 1B) was removed, and the weight element (13 in FIG. 1B) was then released from the top of the mandrel and slowly slid down to the top of the collagen fibers (FIG. 1B).

The hydrated fibers were partially dehydrated (FIG. 1C) to remove the excess solution, while the mandrel was linked to the commercial digital stirrer (12 of FIG. 1B) with a slow rotation speed of about 50-100 RPM to form a thick fiber matrix of aligned fibers with a width of about 1.5 cm. Afterward, the mandrel was removed. As the base of the housing element (FIG. 1D) was permeable to liquid, the weight element would continue to dehydrate the collagen fibers until it hit the fixed height, forming a meniscus-like dense matrix with defined density. The dehydrated dense matrix was subject to a second mold (FIG. 1E) using a different mold to redistribute the collagen materials for enhancing the density on the outer rim of the matrix for higher suture retention strength.

The significantly dehydrated but still wet collagen fiber scaffold was then freeze-dried in a commercial freeze dryer (Virtis, Gardiner, NY). The freeze-dried scaffold with circumferentially oriented fibers was then chemically crosslinked with a vapor of a low molecular weight aldehyde compound (e.g., formaldehyde or glycol-aldehyde), generated from the solution at a concentration of 1-3% at room temperature in a cross-linking tank. Formaldehyde is a common chemical used to crosslink porous collagen-based products. For porous collagen scaffold implants, vapor crosslinking can be used for vapor crosslinking to preserve the three-dimensional structure of the design. Formaldehyde is a small molecule with high vapor pressure at room temperature for crosslinking proteins such as collagen to maintain the three-dimensional size, shape, and dimension of the original design. The disadvantage of formaldehyde crosslinking is that the residual formaldehyde must be removed after crosslinking since it is toxic to cells. Even though most of the formaldehyde is removed by water rinse and is acceptable for implantation, in vitro cell culture studies (a static culture system) often showed residual cell-toxicity effects. Thus, in in vitro cell culture studies, one should eliminate any potential side effects that can affect the cell behavior. It was found that a Tris solution rinse followed by a water rinse is more effective than the water alone and can lower the formaldehyde residuals below 1 ppm, a non-cytotoxicity level to cells. Suitable Tris solutions include, e.g., Tris base, Tris acid, Tris-acetate-EDTA (TEA) buffer and Tris-Borate-EDTA (TBE) buffer. As shown in FIG. 1F, formaldehyde was used for crosslinking the scaffold implants, and used water and a Tris solution to remove the formaldehyde residual. The crosslinked fibers were then cut in half to form two individual meniscus implants.

Similar to formaldehyde, glycolaldehyde can be used as a vapor crosslinker, so the shape and form of the implant can be maintained. Glycolaldehyde has the advantage over formaldehyde in that glycol-aldehyde is much less cytotoxic than formaldehyde. The residual glycolaldehyde molecules do not produce any significant cytotoxicity and are a better crosslinking agent for porous collagen-based scaffolds for in vitro cell culture studies.

To reduce the surface friction, hyaluronic acid (HA), a major component of the synovial fluid, was applied to the interstitial space post-engineering described above. The molecular weights of hyaluronic acid used ranged from about 0.1×10⁶ to about 3×10⁶ Daltons. 100 μl hyaluronic acid of 1-2% (w/v) was first loaded into a 30G needle syringe. The needle was inserted into a fixed position from the back (the thick side) of the meniscus implant, and the hyaluronic acid was injected into the interstitial (intrafibrillar) space. This procedure was repeated 3-5 times at a fixed distance (about 3 mm apart) along the circumferential position and at a height from the bottom of about 2-3 mm. The hyaluronic acid-impregnated implant was dried. Each molecular weight hyaluronic acid was applied to the meniscus implant to provide a prolonged release based on different diffusion rates of the molecules. The total amount of hyaluronic acid loaded was about 0.5-2% of the weight of the meniscus implant, e.g., from 0.5-1%.

Alternatively, hyaluronic acid can be incorporated into scaffolds by immersing the scaffolds in a hyaluronic acid solution containing single or multiple hyaluronic acid molecules with different molecular weights. Hyaluronic acid can be more effectively introduced into the interfibrillar space by applying a vacuum to the scaffold in the solution to facilitate hyaluronic acid incorporation.

To facilitate the speed of healing, autologous bioactive elements or the like can be applied to the scaffold implant at the point of surgical implantation. This can be done via an injection technique. Autologous bioactive elements that can be used include platelet-rich plasma (PRP), isolated from the patient's blood, bioactive components isolated from the patient's bone marrow, recombinant human growth factors, various bioactive macromolecules, differentiated cells, and stem cells. Other methods of incorporating bioactive elements into the scaffold implant include soaking the scaffold with bioactive element solutions and culturing the scaffold with various types of cells prior to implantation.

The above-described scaffold implants can be used to guide the new tissue regeneration of segmental defects in injured or diseased meniscus. It is important that the vascular portion of the defect is preserved and maintained such that autogenous cells and blood vessels can be infiltrated into the porous implant post-surgery during the healing period.

### Example 2

The following characterization studies were performed to compare the current design and that designed by ReGen Biologics (ReGen Equivalent). That was engineered by the current inventors according to the specifications derived from the implant manufactured by ReGen Biologics.

### Density

A meniscus scaffold implant was dried in a desiccator under P₂O₅ overnight and weighed to obtain the dry weight of the scaffold matrix. The dry scaffold was then immersed in 100 ml of water in a beaker for 10 minutes. The surface water was removed with a lint-free cloth. The hydrated scaffold was then weighed. The volume of the scaffold was taken as the sum of the volume occupied by the water and the volume occupied by the collagen, where the volume occupied by the collagen was based on the density of collagen of 1.41 g/cm³ (Haruhiko NODA, The Journal of Biochemistry, Volume 71, Issue 4, April 1972, Pages 699-703). The average density of the scaffold implant was calculated as the ratio of the dry weight of the scaffold and the total volume of the scaffold in g/cm³. The average density of the meniscus scaffold implant was in the range from 0.14 g/cm³ to 0.30 g/cm³, depending on the amount of collagen present. The compartmental density of the inner rim was in a range from 0.15 g/cm³ to 0.35 g/cm³. The compartmental density of the outer rim was in a range from 0.1 g/cm³ to 0.25 g/cm³. Table I summarizes the density comparison of a simulated ReGen collagen meniscus implant of the initial random fiber orientation to the current invention.

TABLE 1
Density

| | ReGen Equivalent | 2-Step Molded Implant |
|---|---|---|
| Average Density (g/cm³) | 0.14 ± 0.01 | 0.19 ± 0.01 |

| | Inner Rim | Outer Rim | Inner Rim | Outer Rim |
|---|---|---|---|---|
| Compartmental | 0.19 ± 0.02 | 0.12 ± 0.01 | 0.26 ± 0.01 | 0.15 ± 0.01 |
| Density | | | | |
| (g/cm³) | | | | |

### Pore Sizes and Pore Volume

The pore sizes were determined from the SEM micrographs at Stevens Institute of Technology via a contract service agreement. Two cross-sections, radial and horizontal, were used. The pore size of each pore was determined as the maximum distance across a pore. The range of pore size is between 50 μm to 200 μm.

The percent (%) pore volume is defined as the empty space (wet weight minus the volume of collagen) divided by the total volume. The pore volume of the scaffold falls within 75% to 90%. The table below summarizes the pore sizes and the pore volume of the ReGen Equipment implant and the implant of the current invention.

TABLE 2
Pore Sizes and Pore volume

| | | | 2-Step Molded | |
|---|---|---|---|---|
| | ReGen Equivalent | | Implant | |
| | Inner Rim | Outer Rim | Inner Rim | Outer Rim |
| Pore Size (μm) | 60.6 ± 26.1 | 74.2 ± 24.9 | 63.9 ± 20.9 | 62.0 ± 21.2 |
| Pore Volume | 90.8 ± 0.5 | | 86.8 ± 0.6 | |
| (%) | | | | |

### Fiber Orientation

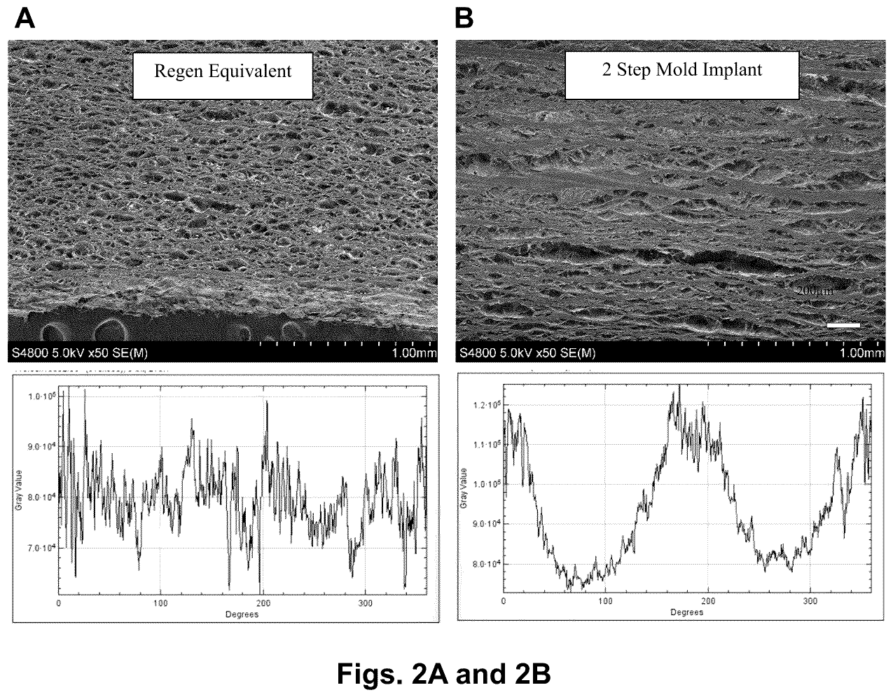

The fiber orientation analysis was provided by the ImageJ software program from the National Institute of Health, applying fast Fourier Transform (FFT) to the SEM images shown in FIG. 2. The data showed that the fibers were longitudinally oriented (FIG. 2B) within an angle of 180 degrees whereas the randomly oriented fibers (FIG. 2A) showed no preferred fiber orientation.

When calculating the degree of fiber alignment from 150 to 210 degrees, the two-step molded implant had 68% of fibers aligned within a 60-degree angle in the circumferential direction where ReGen Equivalent only had 26% aligned. When the angle is covered to 100 degrees (from 130 to 230 degrees), the 2-step molded implant reached 89% fiber alignment and ReGen Equivalent still remained low at 39% fiber alignment.

TABLE 3
Degree of Fiber Alignment

| | | Degree | | | | | | | | |
|---|---|---|---|---|---|---|---|---|---|---|
| | | 90-110 | 110-130 | 130-150 | 150-170 | 170-190 | 190-210 | 210-230 | 230-250 | 250-270 |
| Degree of | ReGen | 20 | 10 | 10 | 30 | 30 | 40 | 30 | 20 | 10 |
| Alignment | Equivalent | | | | | | | | | |
| (%) | 2-Step | 0 | 20 | 30 | 80 | 100 | 80 | 40 | 20 | 20 |
| | Molded | | | | | | | | | |
| | Implant | | | | | | | | | |

### Biomechanical Properties

Tensile strength was determined by the Chatillon mechanical tester (Berwyn, PA). A uniform sample with a dimension of 5 mm×2 mm×10 mm (W×H×L) was cut. The sample was hydrated in 10 ml of water for 10 minutes and fixed at the bottom and top grips. It was pulled at a rate of 2.5 cm per minute until the sample broke into two separate pieces. The ultimate tensile strength was recorded at about 300 N/cm² to 600 N/cm². The compartmental tensile strength was about 300 N/cm² at the outer rim and greater than 600 N/cm² at the inner rim. The randomly oriented fibers (ReGen Equipment) had about 50 N/cm² at the outer rim and about 100 N/cm² at the inner rim.

For the suture retention test, the 1 cm radial segment of the scaffold was cut and hydrated in 10 ml of water for 10 minutes. A 3-0 suture was passed 2 mm from the backside of the scaffold, and a loop knot was formed. The suture loop was attached to a hook of the tester, and the other end was attached to a grip. The sample was pulled at a rate of 2.5 cm/minute until the suture was pulled out. The suture pull-out strength was recorded at 30 N vs. 17 N for the randomly oriented fibers.

TABLE 4
Biomechanical Properties

| | ReGen Equivalent | | 2-Step Molded Implant | |
|---|---|---|---|---|
| | Inner Rim | Outer Rim | Inner Rim | Outer Rim |
| Tensile | 104.5 ± 20.2 | 52.2 ± 22.8 | 720.0 ± 103.5 | 234.5 ± 74.8 |
| Strength | | | | |
| (N/cm²) | | | | |
| Suture | 17.0 ± 1.4 | | 30.6 ± 4.9 | |
| Retention | | | | |
| (N) | | | | |

### Hydrothermal Stability

Hydrothermal stability or hydrothermal shrinkage temperature (Ts) was determined from a differential scanning calorimeter (DSC, Mettler Toledo, Switzerland). The endothermic peak is defined as the shrinkage temperature, in ° C., of the specimen. The Ts was around 67ºC.

TABLE 5
Hydrothermal Stability

| | ReGen | 2-Step Molded |
|---|---|---|
| | Equivalent | Implant |
| Hydrothermal | 67.1 ± 0.1 | 67.0 ± 0.5 |
| Stability (° C.) | | |

### Surface Friction and Durability

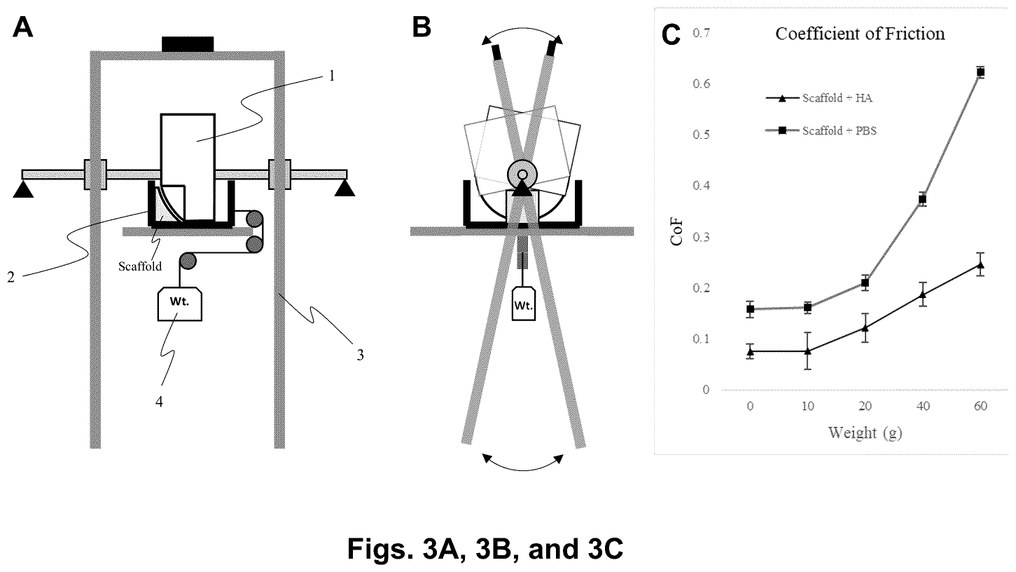

The improvement of surface friction was tested with a device constructed in-house to simulate the knee joint mechanics. A rotational disk (1 in FIG. 3A) is attached to a pendulum (3 in FIG. 3A). The pendulum is linked to a sample housing unit (2 in FIG. 3A) that is closely associated with the rotational disk for the frictional testing. A weight element (4 in FIG. 3A) is attached to the sample housing so that a weight can be applied to the sample, which in turn is transmitted to the rotational disk. A fixed weight is applied to the rotational disk, and the pendulum is set to swing at a fixed angle, which initiates the rotational disk to rotate on the surface of the sample (FIG. 3B). The system is immersed in a water chamber to allow the hyaluronic acid to diffuse to the surface to improve the surface frictional property. The number of vibrations of the pendulum per cycle defines the relative friction of the scaffold. The number of pendulum swings within a defined angle and time were recorded. The scaffolds with and without the hyaluronic acid were tested, and the data were compared. The friction coefficient was calculated according to that published (Crisco, J J, et al., 2007, Proc. Inst. Mech. Eng. H, 221:325-333). The friction coefficient for the collagen surface in PBS solution (FIG. 3C) was in the range of 0.17 to 0.6 (applied force from 0 g to 60 g). The friction coefficient for the collagen surface in a 0.2% hyaluronic acid solution (FIG. 3C) was in the range of 0.07 to 0.24 (applied force from 0 g to 60 g). This preliminary test indicated that a surface lubricated with HA significantly reduces surface friction.

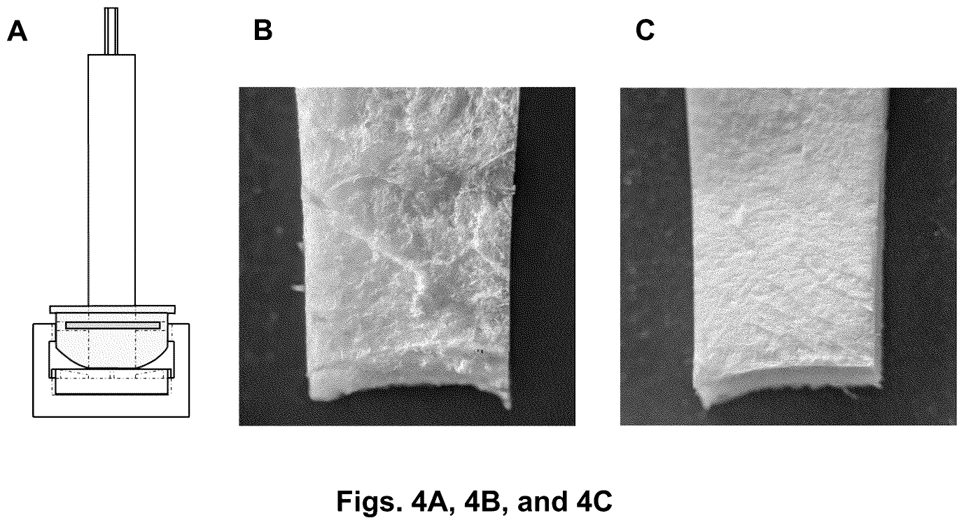

A modified system of the above is used for durability testing (FIG. 4). The duration of time with a fixed weight and RPM was tested. Samples in PBS (FIG. 4B) vs. in the hyaluronic acid solution (FIG. 4C) were examined by light microscope at the conclusion of the testing. The results demonstrated that HA protected the surface and reduced friction.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

What is claimed is:

1. A scaffold implant for repairing injured or diseased meniscus, comprising biopolymer fibers formed of a biopolymer impregnated with glycosaminoglycan,
wherein greater than 30% by weight of the biopolymer fibers are oriented along a circumferential direction, and
wherein the scaffold implant has one or more or all of the following characteristics comprising:
an average density from 0.14 g/cm³ to 0.3 g/cm³,
a compartmental density of an inner rim ranging from 0.15 g/cm³ to 0.35 g/cm³,
a compartmental density of an outer rim ranging from 0.1 g/cm³ to 0.25 g/cm³,
a pore volume from 60% to 90%, and
a surface friction coefficient from 0.05 to 2.0.

2. The scaffold implant of claim 1, wherein the scaffold implant has one or more or all of the following characteristics:
an inner rim tensile strength of 500 N/cm² to 1000 N/cm²,
an outer rim tensile strength of 100 N/cm² to 500 N/cm²,
an inner rim pore size of 40 μm to 150 μm,
an outer rim pore size of 50 μm to 250 μm,
an inner rim pore volume of 70% to 85%,
an outer rim pore volume of 80% to 95%, and
a suture retention of 20 N to 40 N.

3. The scaffold implant of claim 1, wherein the biopolymer comprises collagen.

4. The scaffold implant of claim 3, wherein the collagen comprises a type I collagen, a type II collagen, a type III collagen, or a combination thereof.

5. The scaffold implant of claim 4, wherein the collagen is derived from a human or a non-human animal, or generated from a genetically modified organism.

6. The scaffold implant of claim 1, wherein the glycosaminoglycan is hyaluronic acid, chondroitin sulfate, chitosan, alginic acid, or a combination thereof.

7. The scaffold implant of claim 6, wherein the scaffold implant comprises about 0.1% to about 5% by weight of hyaluronic acid.

8. The scaffold implant of claim 6, wherein the scaffold implant comprises about 0.5% to about 3% by weight of hyaluronic acid.

9. The scaffold implant of claim 8, wherein a molecular weight of the hyaluronic acid ranges from about 0.1×10⁶ Daltons to about 3.0×10⁶ Daltons.

10. The scaffold implant of claim 6, wherein the hyaluronic acid is impregnated into the scaffold implant via injection or soaking in a solution of the hyaluronic acid after a scaffold of the scaffold implant has been engineered.

11. The scaffold implant of claim 1, wherein greater than 50%, 60%, or 70% of the biopolymer fibers are oriented along a circumferential direction.

12. The scaffold implant of claim 1, wherein the friction coefficient is from about 0.1 to about 1.0.

13. The scaffold implant of claim 1, wherein the scaffold implant is crosslinked by an aldehyde-based crosslinking agent.

14. The scaffold implant of claim 13, wherein the aldehyde-based crosslinking agent comprises formaldehyde.

15. The scaffold implant of claim 13, wherein residuals of the aldehyde-based crosslinking agent are removed by water or a Tris solution.

16. The scaffold implant of claim 1, wherein the scaffold implant further comprises one or more bioactive elements.

17. The scaffold implant of claim 16, wherein the bioactive elements are autologous or allogenous.

18. The scaffold implant of claim 16, wherein the bioactive elements comprise platelet rich plasma (PRP), cells, bioactive molecules, or a combination thereof.

19. The scaffold implant of claim 16, wherein the bioactive elements are allogenous and comprise bioactive molecules or stem cells.

20. The scaffold implant of claim 16, wherein the bioactive elements comprise a human recombinant bioactive molecule or a drug.

21. A method for making a scaffold implant, comprising:
preparing a dispersion comprising a biopolymer;
reconstituting the biopolymer in the dispersion into biopolymer fibers;
aligning the biopolymer fibers onto a rotating mandrel to form aligned biopolymer fibers;
dehydrating the biopolymer fibers partially in a dehydration chamber;
placing the aligned biopolymer fibers in a first mold of a defined dimension;
applying a first weight to the biopolymer fibers to dehydrate the biopolymer fibers and form a first partially dehydrated biopolymer scaffold matrix;
removing the first weight and applying a second weight to the first partially dehydrated biopolymer scaffold matrix using a second mold to control a regional density of the biopolymer fibers and form a second partially dehydrated biopolymer scaffold matrix;
freeze-drying the second partially dehydrated biopolymer scaffold matrix to obtain a freeze-dried scaffold matrix;
crosslinking the freeze-dried scaffold matrix; and
sizing the freeze-dried scaffold matrix to obtain a scaffold implant.

22. The method of claim 21, further comprising incorporating hyaluronic molecules into the scaffold implant after the step of sizing.

23. The method of claim 21, wherein the biopolymer comprises collagen.

24. The method of claim 23, wherein the collagen comprises a type I collagen, a type II collagen, a type III collagen, or a combination thereof.

25. A meniscus scaffold implant prepared according to the method of claim 21.

26. A method of treating a joint of a subject using a scaffold implant, comprising providing a scaffold implant according to claim 1, and placing the scaffold implant to the joint of the subject.
